(19) United States
(12) Patent Application Publication
(10) Pub. No.: US 2009/0222096 A1
(43) Pub. Date: Sep. 3, 2009

(54) MULTI-COMPARTMENT EXPANDABLE DEVICES AND METHODS FOR INTERVERTEBRAL DISC EXPANSION AND AUGMENTATION

(75) Inventor: Hai H. TRIEU, Cordova, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/038,953

(22) Filed: Feb. 28, 2008

(51) Int. Cl. A61F 2/44

(52) U.S. Cl. 623/17.16

(57) ABSTRACT

A method of augmenting the nucleus pulposus of an intervertebral disc comprises forming a passage through an annulus fibrosus surrounding the nucleus pulposus and inserting a space creating device comprising a plurality of chambers. Without removing a portion of the nucleus pulposus, plurality of chambers are filled to expand the space creating device to create a space within the nucleus pulposus. The method further comprises injecting at least one biocompatible material into the space within the nucleus pulposus.

### BACKGROUND

Within the spine, the intervertebral disc functions to stabilize and distribute forces between vertebral bodies. The intervertebral disc comprises a nucleus pulposus which is surrounded and confined by the annulus fibrosus. Intervertebral discs are prone to injury and degeneration. For example, herniated discs typically occur when normal wear, or exceptional strain, causes a disc to rupture. Degenerative disc disease typically results from the normal aging process, in which the tissue gradually loses its natural water and elasticity, causing the degenerated disc to shrink and possibly rupture.

Intervertebral disc injuries and degeneration are frequently treated by replacing or augmenting the existing disc material. Current methods and instrumentation used for treating the disc require a relatively large hole to be cut in the disc annulus to allow introduction of the implant. After the implantation, the large hole in the annulus must be plugged, sewn closed, or other wise blocked to avoid allowing the implant to be expelled from the disc. Besides weakening the annular tissue, creation of the large opening and the subsequent repair adds surgical time and cost. A need exists for devices, instrumentation, and methods for implanting an intervertebral implant using minimally invasive surgical techniques.

### SUMMARY

In one embodiment, a method of augmenting the nucleus pulposus of an intervertebral disc comprises forming a passage through an annulus fibrosus surrounding the nucleus pulposus and inserting a space creating device comprising a plurality of chambers. Without removing a portion of the nucleus pulposus, plurality of chambers are filled to expand the space creating device to create a space within the nucleus pulposus. The method further comprises injecting at least one biocompatible material into the space within the nucleus pulposus.

In another embodiment, a device for supplementing a nucleus pulposus comprises an expandable central body comprising a cylindrical portion bounded by a pair of curved surfaces and adapted to receive a first biocompatible material. At least one of the pair of curved surfaces is adapted to penetrate a vertebral endplate adjacent the nucleus pulposus. The device also comprises an expandable ring member surrounding the cylindrical portion and adapted to receive a second biocompatible material.

In another embodiment, a system for treating a nucleus pulposus of an intervertebral disc comprises a cannula adapted to access an annulus fibrosus of the intervertebral disc and a multi-chamber spacing device comprising at least three inflatable chambers. Each of the inflatable chambers is connected to at least one other of the inflatable chambers and the spacing device is collapsible for passage through the cannula. The system further comprises a catheter connected to the spacing device and extendable through the cannula.

A system for treating a nucleus pulposus of an intervertebral disc comprises a cannula adapted to access an annulus fibrosus of the intervertebral disc and a multi-chamber spacing device comprising two connected and inflatable chambers One of the inflatable chambers is expandable along the annulus fibrosus. The system further comprises a catheter connected to the spacing device and extendable through the cannula.

Additional embodiments are included in the attached drawings and the description provided below.

### BRIEF DESCRIPTION OF THE DRAWINGS

FIG. 1 is a sagittal view of a section of a vertebral column.

FIGS. 2-5 are a sequence of superior views of a nucleus augmentation treatment.

FIG. 6 is a superior view of a nucleus augmentation device implanted in the vertebral column.

FIG. 7. is a sagittal view of the nucleus augmentation device of FIG. 6.

FIG. 8 is a perspective view of a nucleus augmentation device according to another embodiment of the disclosure.

FIG. 9 is a cross-sectional view of the nucleus augmentation device of FIG. 8.

FIGS. 10-18 are superior views of nucleus augmentation devices according to alternative embodiments of the disclosure.

### DETAILED DESCRIPTION

The present disclosure relates generally to methods and devices for augmenting an intervertebral disc, and more particularly, to methods and devices for minimally invasive nucleus augmentation procedures. For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

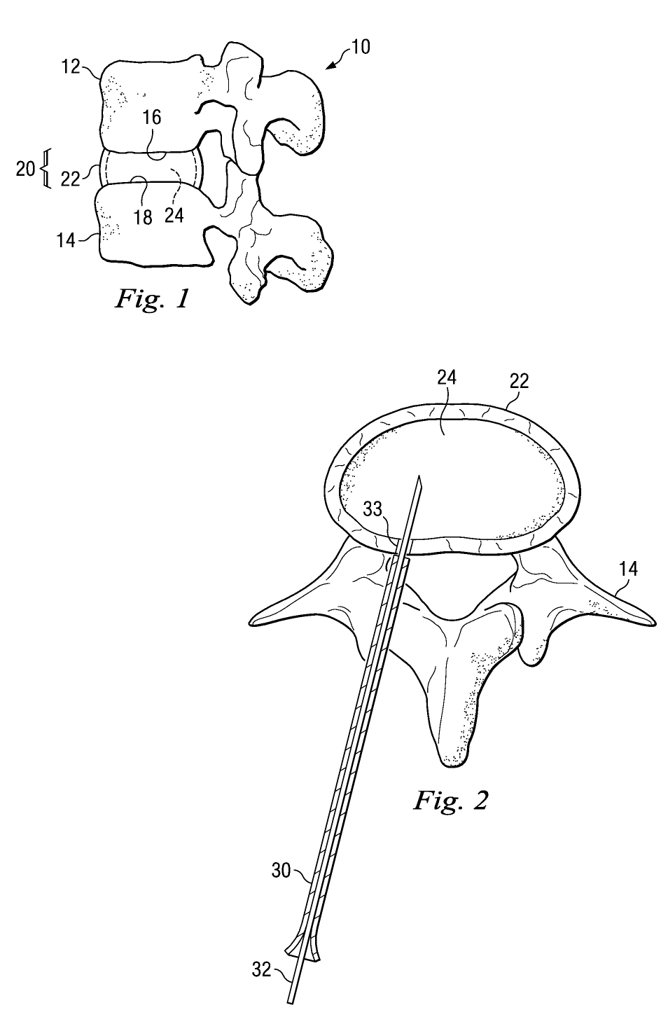

Referring first to FIG. 1, the reference numeral 10 refers to a vertebral joint section or a motion segment of a vertebral column. The joint section 10 includes adjacent vertebral bodies 12, 14. The vertebral bodies 12, 14 include endplates 16, 18, respectively. An intervertebral disc space 20 is located between the endplates 16, 18, and an annulus 22 surrounds the space 20. In a healthy joint, the space 20 contains a nucleus pulposus 24.

Referring now to FIGS. 2-5, in this embodiment, the nucleus 24 may be accessed by inserting a cannula 30 into the patient and locating the cannula at or near the annulus 22. An accessing instrument 32, such as a trocar needle, a K-wire, or a dilator is inserted through the cannula 30 and used to penetrate the annulus 22, creating an annular opening 33. With the opening 33 created, the accessing instrument 32 may be removed and the cannula 30 left in place to provide passageway for additional instruments.

In this embodiment, the nucleus is accessed using a posterolateral approach. In alternative embodiments, the annulus may be accessed with a lateral approach, an anterior approach, a trans-pedicular/vertebral endplate approach or any other suitable nucleus accessing approach. Although a unilateral approach is described, a multi-lateral approach may be suitable. For example, a suitable bilateral approach to nucleus augmentation may involve a combination approach including an annulus access opening and an endplate access opening.

It is understood that any cannulated instrument including a guide needle or a trocar sleeve may be used to guide the accessing instrument.

In this embodiment, the natural nucleus, or what remains of it after natural disease or degeneration, may remain intact with no tissue removed. In alternative embodiments, partial or complete nucleotomy procedures may be performed.

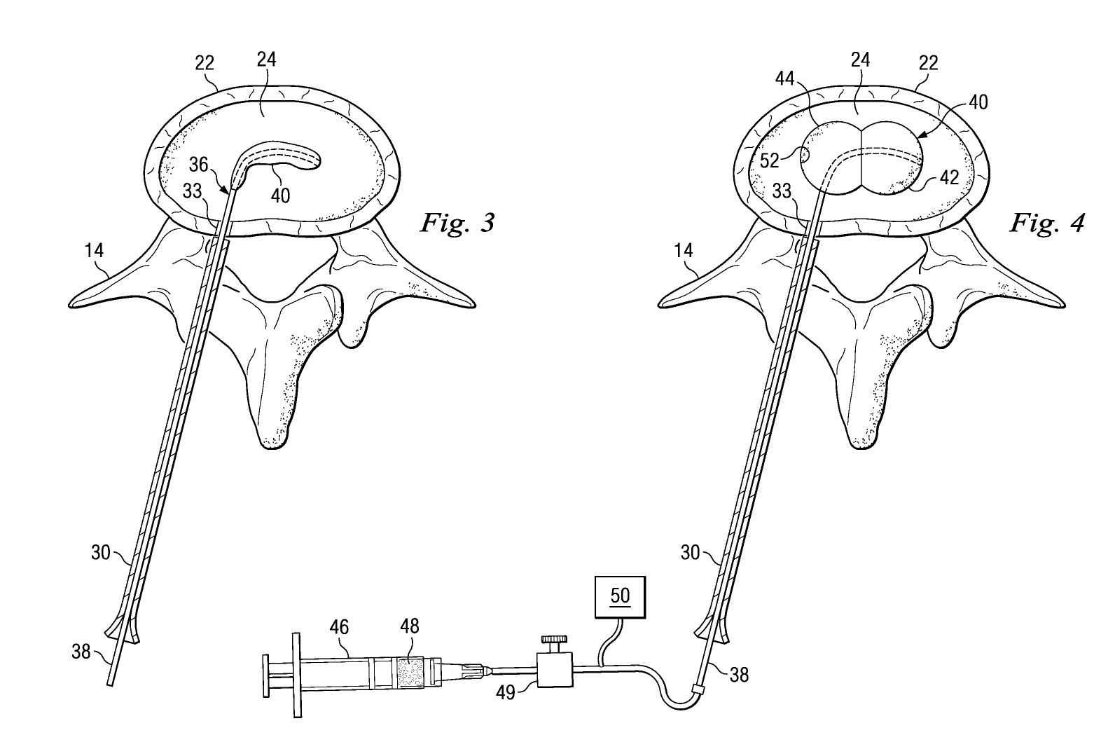

As shown in FIG. 3, a space creating device 36 having a catheter portion 38 and a multi-compartment or multi-chamber spacing portion 40 may be inserted through the cannula 30 and the annular opening 33 into the nucleus 24. In this embodiment, the multi-compartment spacing portion 40 is a multi-compartment expandable device such as a balloon which may be formed of elastic or non-elastic materials. The space creating device 36 may be rolled or folded to minimize its size for insertion through the cannula 30.

The balloon can be of various shapes including conical, spherical, square, long conical, long spherical, long square, tapered, stepped, dog bone, offset, or combinations thereof. Balloons can be made of various polymeric materials such as polyethylene terephthalates, polyolefins, polyurethanes, nylon, polyvinyl chloride, silicone, polyetheretherketone, polylactide, polyglycolide, poly(lactide-co-glycoli-de), poly(dioxanone), poly(.epsilon.-caprolactone), poly(hydroxylbutyrate), poly(hydroxylvalerate), tyrosine-based polycarbonate, polypropylene fumarate or combinations thereof. Additionally, the expandable device may be molded or woven.

In alternative embodiments, the space creating device may have multiple catheter portions with each separately feeding a different compartment of the spacing portion.

Referring now to FIG. 4, the multi-compartment spacing portion 40 has two separate or substantially separate but attached lobes or chambers 42, 44. Each of the compartments 42, 44 are connected to the catheter portion 38. The catheter portion 38 is attached to a material delivery device 46, such as a syringe, which may be filled with a biocompatible material 48. The biocompatible material 48 may be pressurized and injected through the catheter portion 38 of the space creating device 36 to pressurize, inflate, and fill the compartments 42, 44 of the spacing portion 40. As the compartments become filled, the spacing portion 40 may unroll or unfold from its minimized configuration. The filling of the spacing portion 40 may be controlled by a control mechanism 49, such as a valve. The control mechanism 49 may control the total volume of the material injected into the spacing portion 40, but may also control the volume of material injected into each of the compartments 42, 44. The inflation medium may be injected under pressure supplied by a hand, electric, or other type of powered pressurization device. The internal balloon pressure may be monitored with a well known pressure gauge 50. The pressure gauge 50 or a pressure limiter may be used to avoid over inflation or excessive injection. The rate of inflation and level of inflation of the spacing portion 40 can be varied between patients depending on disc condition.

As the spacing portion 40 is gradually filled and inflated, the surrounding nucleus tissue may become displaced or stretched, creating a space 52. The inflation may also cause the intradiscal pressure to increase. Both the pressure increase and the direct expansion of the spacing portion 40 may cause the endplates 16, 18 to distract.

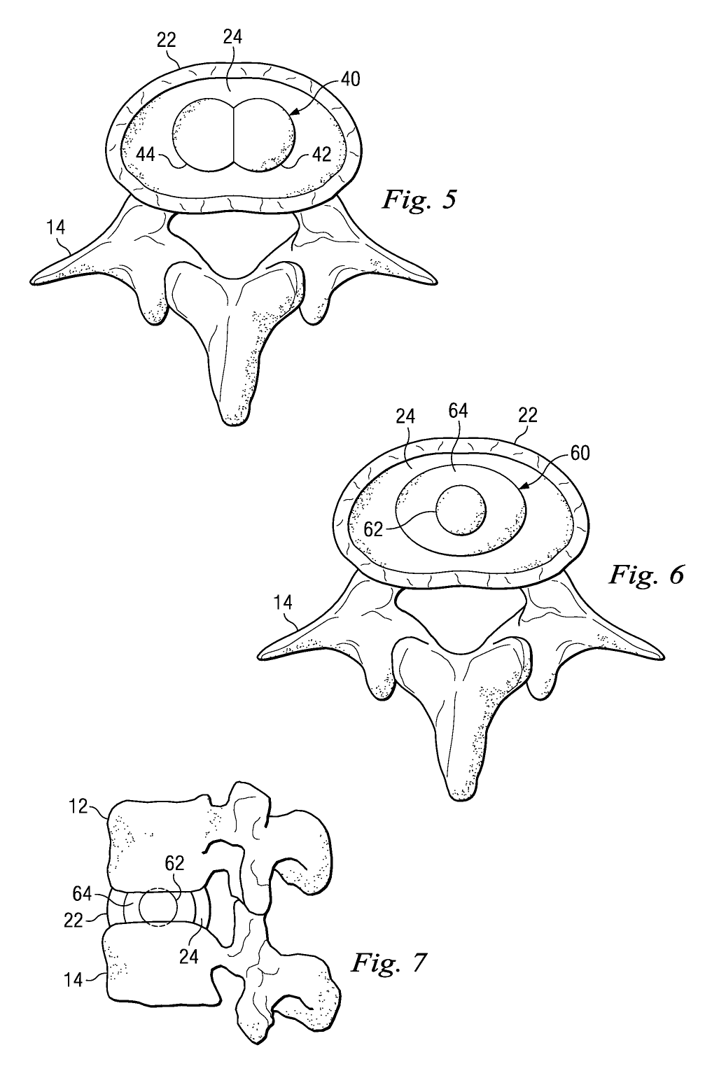

Referring now to FIG. 5, after the spacing portion 40 is inflated to the desired level, the catheter portion 38 is detached from the spacing portion 40 and removed from the patient. If the selected biocompatible material 48 is curable in situ, the catheter portion 38 may be removed during or after curing to minimize leakage. The opening 33 may be small enough, for example less than 3 mm, that it will close or close sufficiently that the spacing portion 40 will remain within the annulus. The use of an annulus closure device such as a suture, a plug, or a material sealant is optional. The cannula 30 may be removed and the minimally invasive surgical incision closed.

Examples of biocompatible materials 48 which may be used for disc augmentation include natural or synthetic and resorbable or non-resorbable materials. Natural materials include various forms of collagen that are derived from collagen-rich or connective tissues such as an intervertebral disc, fascia, ligament, tendon, skin, or demineralized bone matrix. Material sources include autograft, allograft, xenograft, or human-recombinant origin materials. Natural materials also include various forms of polysaccharides that are derived from animals or vegetation such as hyaluronic acid, chitosan, cellulose, or agar. Other natural materials include other proteins such as fibrin, albumin, silk, elastin and keratin. Synthetic materials include various implantable polymers or hydrogels such as silicone, polyurethane, silicone-polyurethane copolymers, polyolefin, polyester, polyacrylamide, polyacrylic acid, polyvinyl alcohol, polyethylene oxide, polyethylene glycol, polylactide, polyglycolide, poly(lactide-co-glycolide), poly(dioxanone), poly(.epsilon.-caprolactone), poly(hydroxylbutyrate), poly(hydroxylvalerate), tyrosine-based polycarbonate, polypropylene fumarate or combinations thereof. Suitable hydrogels may include poly(vinyl alcohol), poly(acrylic acids), poly(methacrylic acids), copolymers of acrylic acid and methacrylic acid, poly(acrylonitrile-acrylic acid), polyacrylamides, poly(N-vinyl-2-pyrrolidone), polyethylene glycol, polyethyleneoxide, polyacrylates, poly(2-hydroxy ethyl methacrylate), copolymers of acrylates with N-vinyl pyrrolidone, N-vinyl lactams, polyurethanes, polyphosphazenes, poly(oxyethylene)-poly(oxypropylene) block polymers, poly(oxyethylene)-poly(oxypropylene) block polymers of ethylene diamine, poly(vinyl acetate), and sulfonated polymers, polysaccharides, proteins, and combinations thereof.

The selected biocompatible material may be curable or polymerizable in situ. The biocompatible material may transition from a flowable to a non-flowable state shortly after injection. One way to achieve this transition is by adding a crosslinking agent to the biomaterial before, during, or after injection. The biocompatible material in its final state may be load-bearing, partially load-bearing, or simply tissue augmenting with minimal or no load-bearing properties.

Proteoglycans may also be included in the injectable biocompatible material 48 to attract and/or bind water to keep the nucleus 24 hydrated. Regnerating agents may also be incorporated into the biocompatible material. An exemplary regenerating agent includes a growth factor. The growth factor can be generally suited to promote the formation of tissues, especially of the type(s) naturally occurring as components of an intervertebral disc. For example, the growth factor can promote the growth or viability of tissue or cell types occurring in the nucleus pulposus, such as nucleus pulposus cells and chondrocytes, as well as space filling cells, such as fibroblasts and connective tissue cells, such as ligament and tendon cells. Alternatively or in addition, the growth factor can promote the growth or viability of tissue types occurring in the annulus fibrosus, as well as space filling cells, such as fibroblasts and connective tissue cells, such as ligament and tendon cells. An exemplary growth factor can include transforming growth factor-β (TGF-β) or a member of the TGF-β superfamily, fibroblast growth factor (FGF) or a member of the FGF family, platelet derived growth factor (PDGF) or a member of the PDGF family, a member of the hedgehog family of proteins, interleukin, insulin-like growth factor (IGF) or a member of the IGF family, colony stimulating factor (CSF) or a member of the CSF family, growth differentiation factor (GDF), cartilage derived growth factor (CDGF), cartilage derived morphogenic proteins (CDMP), bone morphogenetic protein (BMP), or any combination thereof. In particular, an exemplary growth factor includes transforming growth factor β protein, bone morphogenetic protein, fibroblast growth factor, platelet-derived growth factor, insulin-like growth factor, or any combination thereof.

Therapeutic or biological agents may also be incorporated into the biomaterial. An exemplary therapeutic or biological agent can include a soluble tumor necrosis factor α-receptor, a pegylated soluble tumor necrosis factor α-receptor, a monoclonal antibody, a polyclonal antibody, an antibody fragment, a COX-2 inhibitor, a metalloprotease inhibitor, a glutamate antagonist, a glial cell derived neurotrophic factor, a B2 receptor antagonist, a substance P receptor (NK1) antagonist, a downstream regulatory element antagonistic modulator (DREAM), iNOS, a inhibitor of tetrodotoxin (TTX)-resistant Na+-channel receptor subtypes PN3 and SNS2, an inhibitor of interleukin, a TNF binding protein, a dominant-negative TNF variant, Nanobodies™, a kinase inhibitor, or any combination thereof. These regenerating, therapeutic, or biological agents may promote healing, repair, regeneration and/or restoration of the disc, and/or facilitate proper disc function.

In an alternative embodiment, the material delivery device 46 may contain an inflation medium instead of a biocompatible material. The inflation medium may be pressurized and injected through the catheter portion 38 of the space creating device 36 to pressurize and inflate the compartments 42, 44 of the spacing portion 40. The inflation of the spacing portion 40 may be controlled by the control mechanism 49. The inflation medium may be injected under pressure supplied by a hand, electric, or other type of powered pressurization device. The internal balloon pressure may be monitored with the pressure gauge 50. The pressure gauge 50 or a pressure limiter may be used to avoid over inflation or excessive injection. The rate of inflation and level of inflation of the spacing portion 40 can be varied between patients depending on disc condition. The inflation medium may be a saline and/or radiographic contrast medium such as sodium diatrizoate solution sold under the trademark Hypaque by Amersham Health, a division of GE Healthcare (Amersham, UK).

As the spacing portion 40 is gradually inflated, the surrounding nucleus tissue may become displaced or stretched, creating a space within the nucleus pulposus 24. The inflation may also cause the intradiscal pressure to increase. Both the pressure increase and the direct expansion of the spacing portion 40 may cause the endplates 16, 18 to distract.

In this alternative embodiment, the space creating portion 40 may be deflated and removed and the biocompatible material 48 injected into the space formed within the nucleus pulposus 24 and vacated by the space creating portion 40. The material 48 may be injected after the space creating portion 40 has been deflated and removed or may be injected while the space creating portion 40 is being deflated and removed. For example, the biomaterial 48 may become increasingly pressurized while the pressure in the space creating portion 40 is lowered. In some procedures, the material 48 may be injected before the space creating portion 40 is removed. With the material 48 injected and the space creating portion 40 removed, the cannula 30 may be removed and the minimally invasive surgical incision closed.

Any of the steps of the above described methods including expansion of the space creating portion 40 and filling the space created by the space creating portion 40 may be monitored and guided with the aid of imaging methods such as fluoroscopy, x-ray, computed tomography, magnetic resonance imaging, and/or image guided surgical technology such as a Stealth Station surgical navigation system (Medtronic, Inc., Minneapolis, Minn.) or a BrainLab system (Heimstetten, Germany).

In another alternative embodiment, the space creating portion may be inflated with an inflation medium and the inflation medium replaced with a biocompatible material. The space creating portion filled with biocompatible material may be detached from the catheter portion and may remain in the nucleus 24 as an implant.

Alternative space creating portions and space creating methods are described in the currently pending applications “Devices, Apparatus, and Methods for Improved Disc Augmentation” (Attorney Docket No. 31132.512) and “Devices, Apparatus, and Methods for Bilateral Approach to Disc Augmentation” (Attorney Docket No. 31132.513), both filed Apr. 27, 2006 and incorporated herein by reference.

Referring now to FIGS. 6-7, in this embodiment, a multi-chamber spacing portion 60 comprises a central spherical chamber 62 and a ring or donut (torus) chamber 64. The spherical chamber 62 and the ring chamber 64 may be molded together, bonded together, sewn together, or otherwised affixed to one another. The spacing portion 60 may be inserted into the nucleus pulposus and filled using any of the methods described above. The chambers 62, 64 may be independently filled with any of the materials described above. For example, the spherical chamber 62 may be filled with a material that becomes relatively hard such as polymethylmethacrylate (PMMA) bone cement. The ring chamber 64 may be filled with a material that remains relatively soft compared to the PMMA, such as silicone or polyurethane. In this embodiment, the spherical chamber 62 may be inflated first and the ring chamber 64 may inflated after the chamber 62 is inflated. As shown in FIG. 7, after inflation, the upper and lower surfaces of the spherical chamber 62 may extend outward beyond the ring chamber 64. As the central spherical chamber 62 becomes filled and hardens, the upper and lower surfaces of the chamber 62 may penetrate the contacted endplate surfaces of the vertebral bodies 12, 14, securing or anchoring the spacing portion 60 between the two endplates 16, 18. In this embodiment, the spacing portion 60 may function as an anchored distractor. Penetration of the endplate is broadly understood to include piercing of the endplate, indentation of the endplate, deformation of the endplate, remodeling of the endplate over a period of time to conform to the spacing portion, or any other reaction of or change to the endplate as a result of high contract stress with the spacing portion.

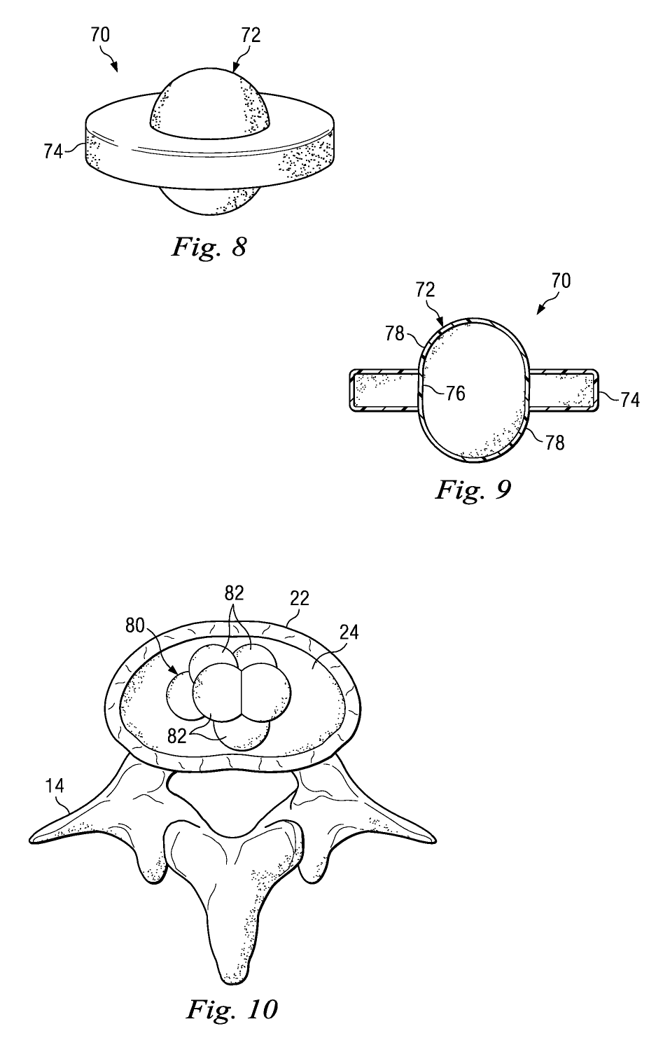

Referring now to FIGS. 8-9, in this embodiment, a multi-chamber spacing portion 70 comprises a central chamber 72 and a ring or donut (torus) chamber 74. The central chamber 72 includes a cylindrical area 76 bounded by curved or domed surfaces 78. The central chamber 72 and the ring chamber 74 may be molded together, bonded together, sewn together, or otherwised affixed to one another. The spacing portion 70 may be inserted into the nucleus pulposus and filled using any of the methods described above. The chambers 72, 74 may be independently filled with any of the materials described above. For example, the central chamber 72 may be filled with a material that becomes relatively hard such as polymethylmethacrylate (PMMA) bone cement. The ring chamber 74 may be filled with a material that remains relatively soft compared to the PMMA, such as silicone or polyurethane. In this embodiment, the central chamber 72 may be inflated first and the ring chamber 74 may inflated after the chamber 72 is inflated. As shown in FIG. 8, after inflation, the curved surfaces 78 of the chamber 72 may extend outward beyond the ring chamber 74. As the central chamber 72 becomes filled and hardens, the upper and lower curved surfaces 78 of the chamber 72 may penetrate the contacted endplate surfaces of the vertebral bodies 12, 14, securing the spacing portion 70 between the two endplates 16, 18. The filled cylindrical area 76 of the central chamber 72 may provide greater axial support to the curved surfaces 78, enhancing penetration of the central chamber into the endplates and resisting migration of the spacing portion 70. Penetration of the endplate is broadly understood to include piercing of the endplate, indentation of the endplate, deformation of the endplate, remodeling of the endplate over a period of time to conform to the spacing portion, or any other reaction of or change to the endplate as a result of high contract stress with the spacing portion.

Referring now to FIG. 10, in this embodiment, a multi-chamber spacing portion 80 comprises multiple clustered lobes 82. The spacing portion 80 may be inserted into the nucleus pulposus and filled using any of the methods described above. The lobes 82 may be selectively filled to compensate for a particular patient's disc degeneration or injury. For example, lobes located in an area of significant disc degeneration may be filled with biocompatible material to restore natural disc height and elasticity. Lobes located closer to intact and hydrated nucleus tissue may be unfilled, underfilled, or filled with a softer material to blend the implant with the natural nucleus. Multiple lobes may provide the physician with greater flexibility in adapting to a particular patient's anatomy.

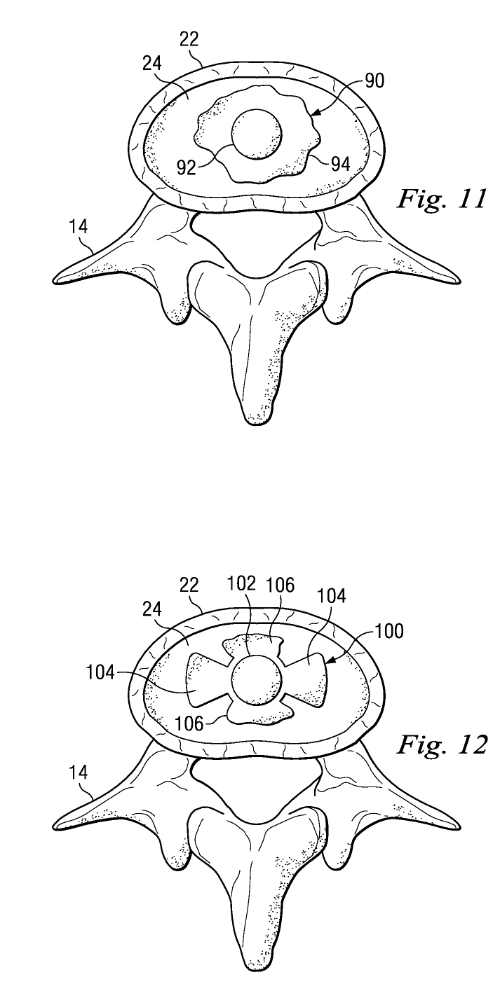

Referring now to FIG. 11, in this embodiment, a multi-chamber spacing portion 90 comprises a central chamber 92 and an irregularly shaped chamber 94. The central chamber 92 may be spherical or cylindrical as in the embodiments described above, although other shapes may be suitable. The chamber 94 is an irregular shape selected to conform to, or compensate for loss in, the surrounding nucleus tissue. The spacing portion 90 may be inserted into the nucleus pulposus and filled using any of the methods described above. The chambers 92, 94 may be independently filled with any of the materials described above. For example, the central chamber 92 may be filled with a material that becomes relatively hard such as polymethylmethacrylate (PMMA) bone cement. The irregular chamber 94 may be filled with a material that remains relatively soft compared to the PMMA, such as silicone or polyurethane. The irregular chamber 94 may be unfilled, underfilled, or filled with a softer material to blend the implant with the natural nucleus. The irregular shape may provide the physician with greater flexibility in adapting to a particular patient's anatomy.

Referring now to FIG. 12, in this embodiment, a multi-chamber spacing portion 100 comprises a central chamber 102 and outer chambers 104, 106. The central chamber 102 may be spherical or cylindrical as in the embodiments described above, although other shapes may be suitable. The outer chambers 104, 106 may be selectively filled to compensate for a particular patient's disc degeneration or injury. For example, chambers 104 may be filled with biocompatible material to restore natural disc function in areas of greater disc degeneration or injury. Chambers 106 may be unfilled or underfilled for areas requiring less augmentation. Multiple chambers may provide the physician with greater flexibility in adapting to a particular patient's anatomy. The spacing portion 100 may be inserted into the nucleus pulposus and filled using any of the methods described above. The chambers 102, 104, 106 may be independently filled with any of the materials described above.

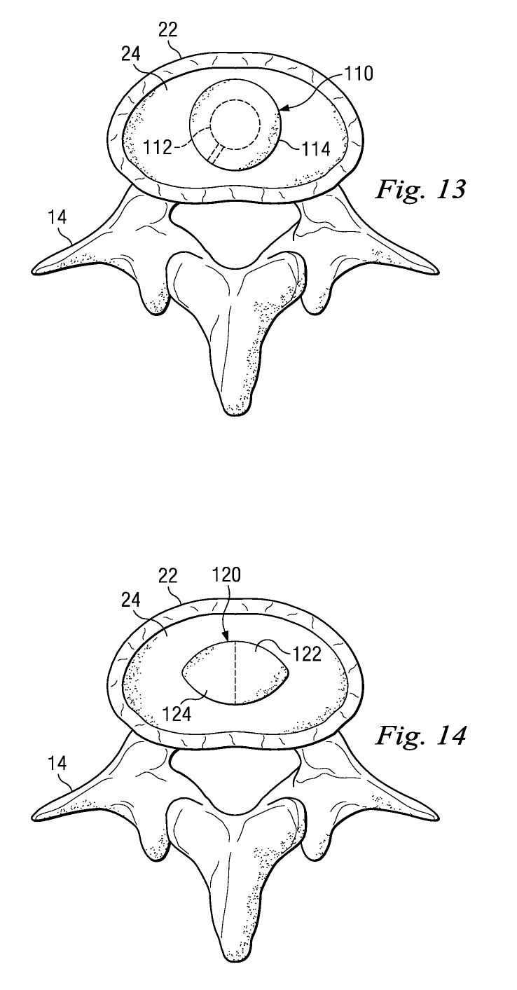

Referring now to FIG. 13, in this embodiment, a multi-chamber spacing portion 110 comprises a spherical central chamber 112 and a spherical outer chamber 114, concentric with central chamber 112. Although the chambers 112, 114 are described as spherical, other configurations may be suitable. The spacing portion 110 may be inserted into the nucleus pulposus and filled using any of the methods described above. The chambers 112, 114 may be independently filled with any of the materials described above. For example, the central chamber 112 may be filled with a material that becomes relatively hard such as polymethylmethacrylate (PMMA) bone cement. The irregular chamber 114 may be filled with a material that remains relatively soft compared to the PMMA, such as silicone or polyurethane.

Referring now to FIG. 14, in this embodiment, a multi-chamber spacing portion 120 has a fusiform structure similar to a football. Other shapes such as ellipsoid may also be suitable. The spacing portion 120 includes chambers 122, 124. The spacing portion 120 may be inserted into the nucleus pulposus and filled using any of the methods described above. The chambers 122, 124 may be independently filled with any of the materials described above. For example, the chambers 122, 124 may both be filled with polyurethane materials, however the chamber 122 may be underfilled or filled with a different type of polyurethane having a final hardness lower than that used for chamber 124. In this way, the spacing portion 120 may be tailored toward a particular patient's anatomy.

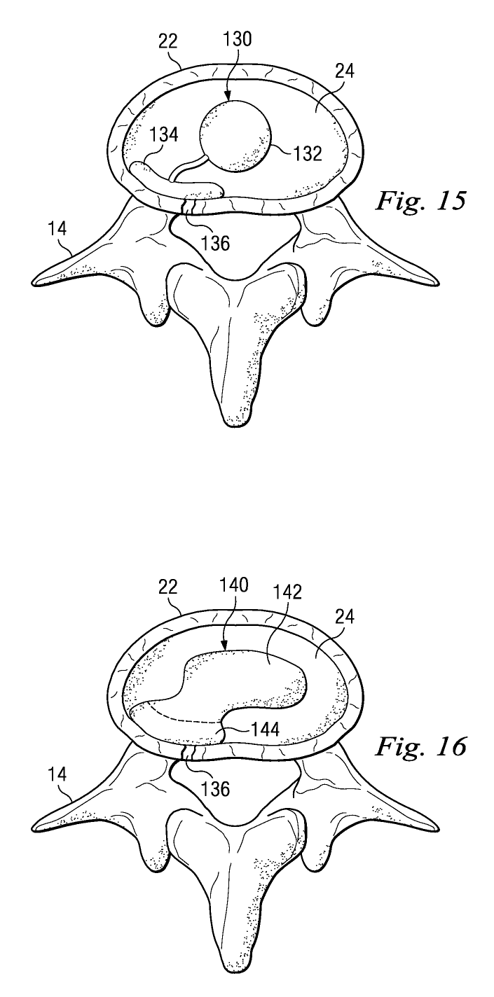

Referring now to FIG. 15, in this embodiment, a multi-chamber spacing portion 130 comprises a spherical central chamber 132 and an outer chamber 134 extending along the annulus 22 to occlude an annulus defect 136. Although the chamber 132 is described as spherical, other configurations may be suitable. The spacing portion 130 may be inserted into the nucleus pulposus and filled using any of the methods described above. The chambers 132, 134 may be independently filled with any of the materials described above. For example, the central chamber 132 may be filled with a material that becomes relatively hard such as polymethylmethacrylate (PMMA) bone cement. The outer occluding chamber 134 may be filled with a material that also becomes relatively hard to prevent the migration of chamber 132 through the defect 136.

Referring now to FIG. 16, in this embodiment, a multi-chamber spacing portion 140 comprises an irregularly shaped central chamber 142 and an outer chamber 144 extending along the annulus 22 to occlude an annulus defect 136. The spacing portion 140 may be inserted into the nucleus pulposus and filled using any of the methods described above. The chambers 142, 144 may be independently filled with any of the materials described above. For example, the central chamber 142 may be filled with a material that becomes relatively compliant or soft. The outer occluding chamber 144 may be filled with a material that also becomes relatively hard to prevent the migration of chamber 142 through the defect 136.

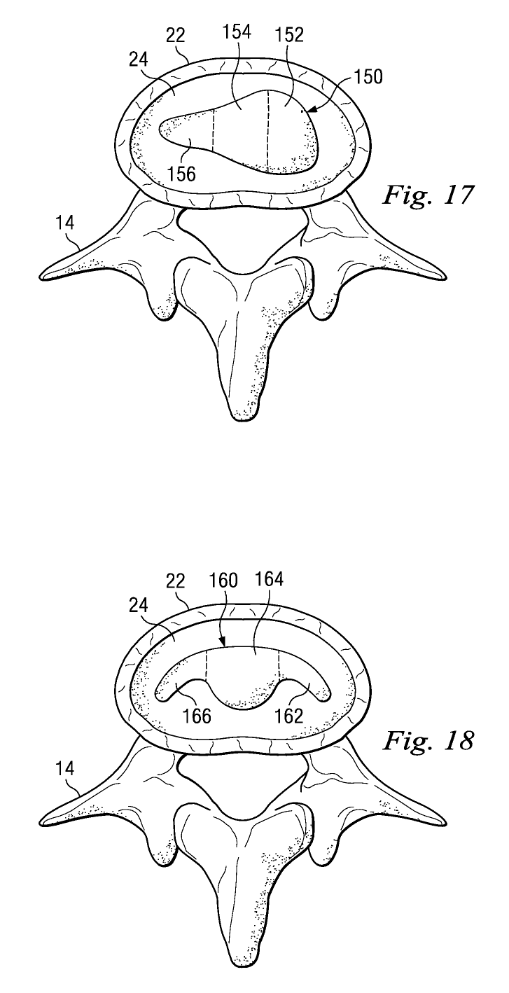

Referring now to FIG. 17, in this embodiment, a multi-chamber spacing portion 150 comprises three chambers 152, 154, 156, serially arranged. The spacing portion 150 may be inserted into the nucleus pulposus and filled using any of the methods described above. The chambers 152, 154, 156 may be independently filled with any of the materials described above. The chambers 152, 154, 156 may also be filled, underfilled, or unfilled to achieve a desired result for a particular patient. The shape and number of the chambers depicted is merely exemplary and other shapes, configuration, and quantities of chambers may be suitable.

Referring now to FIG. 18, in this embodiment, a multi-chamber spacing portion 160 comprises three chambers 162, 164, 166, serially arranged. The spacing portion 160 may be inserted into the nucleus pulposus and filled using any of the methods described above. The chambers 162, 164, 166 may be independently filled with any of the materials described above. The chambers 162, 164, 156 may also be filled, underfilled, or unfilled to achieve a desired result for a particular patient. The shape and number of the chambers depicted is merely exemplary and other shapes, configuration, and quantities of chambers may be suitable.

As used in this description, the term “filled” should be broadly construed describe those chambers that are not only completely filled, but also partially filled. It is understood that some chambers of a filled multi-chamber space creating device may be unfilled or partially filled.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” “right,” “anterior,” “posterior,” “superior,” “inferior,” “upper,” and “lower” are for illustrative purposes only and can be varied within the scope of the disclosure. In the claims, means-plus-function clauses are intended to cover the elements described herein as performing the recited function and not only structural equivalents, but also equivalent elements.

What is claimed is:

1. A method of augmenting the nucleus pulposus of an intervertebral disc, the method comprising:
forming a passage through an annulus fibrosus surrounding the nucleus pulposus;
inserting a space creating device comprising a plurality of chambers;
without removing a portion of the nucleus pulposus, filling the plurality of chambers to expand the space creating device to create a space within the nucleus pulposus; and
injecting at least one biocompatible material into the space within the nucleus pulposus.

2. The method of claim 1 further comprising:
removing the space creating device from the nucleus pulposus.

3. The method of claim 1 wherein the plurality of chambers are a plurality of clustered lobes.

4. The method of claim 3 wherein the step of injecting further comprises filling one of the plurality of clustered lobes less than another of the plurality of clustered lobes.

5. The method of claim 1 wherein the step of injecting comprises filling an outer ring chamber and a central chamber of the space creating device with the at least one biocompatible material, wherein the central chamber comprises a cylindrical body bounded by a pair of curved surfaces.

6. The method of claim 5 wherein the at least one biocompatible material comprises first and second biocompatible materials and the step of filling comprising filling the central chamber with the first biocompatible material and filling the outer ring chamber with the second biocompatible material, wherein the first biocompatible material is harder than the second biocompatible material.

7. The method of claim 5 further comprising anchoring at least one of the curved surfaces into a vertebral endplate adjacent the intervertebral disc.

8. The method of claim 1 wherein the step of injecting comprises filling an outer ring chamber and a central chamber of the space creating device with the at least one biocompatible material, wherein the central chamber is spherical and includes a curved surface adapted to extend beyond the outer ring chamber to penetrate a vertebral endplate adjacent to the intervertebral disc.

9. The method of claim 1 wherein the space creating device is fusiform shaped.

10. The method of claim 1 wherein the space creating device is ellipsoid.

11. The method of claim 1 wherein the space creating device comprises an annular occlusion chamber.

12. The method of claim 1 wherein the space creating device comprises at least three serially connected chambers.

13. The method of claim 1 wherein the step of expanding the space creating device further comprises unrolling the space creating device within the nucleus pulposus.

14. The method of claim 1 wherein the at least one biocompatible material is curable in-situ.

15. The method of claim 1 wherein the at least one biocompatible material is polymerizable in-situ.

16. A device for supplementing a nucleus pulposus comprising:
an expandable central body comprising a cylindrical portion bounded by a pair of curved surfaces and adapted to receive a first biocompatible material, wherein at least one of the pair of curved surfaces is adapted to penetrate a vertebral endplate adjacent the nucleus pulposus and
an expandable ring member surrounding the cylindrical portion and adapted to receive a second biocompatible material.

17. The device of claim 16 wherein the first biocompatible material has a hardness measurement greater than the second biocompatible material.

18. The device of claim 16 wherein the second biocompatible material has a hardness measurement greater than the first biocompatible material.

19. The device of claim 16 wherein the expandable ring member is attached to the expandable central body.

20. The device of claim 16 wherein the first biocompatible material is polymethylmethacrylate.

21. The device of claim 16 wherein the second biocompatible material is silicone.

22. The device of claim 16 wherein the expandable central body and ring member are adapted to pass through an opening in an annulus fibrosus to supplement the nucleus pulposus, wherein the nucleus pulposus is unresected.

23. The device of claim 16 wherein the first biocompatible material is curable in-situ.

24. The device of claim 16 wherein the central body is affixed to the ring member.

25. A device for supplementing a nucleus pulposus comprising:
an expandable central body comprising a spherical portion, including a pair of curved surfaces, and adapted to receive a first biocompatible material, wherein at least one of the pair of curved surfaces is adapted to penetrate a vertebral endplate adjacent the nucleus pulposus and
an expandable ring member encircling the central body and adapted to receive a second biocompatible material.

26. The device of claim 25 wherein the first biocompatible material is curable in-situ.

27. The device of claim 25 wherein the second biocompatible material is curable in-situ.

28. The device of claim 25 wherein the first biocompatible material is harder than the second biocompatible material.

29. The device of claim 25 wherein the central body is affixed to the ring member.

30. A system for treating a nucleus pulposus of an intervertebral disc, the system comprising:
a cannula adapted to access an annulus fibrosus of the intervertebral disc;
a multi-chamber spacing device comprising at least three inflatable chambers, wherein each of the inflatable chambers is connected to at least one other of the inflatable chambers and the spacing device is collapsible for passage through the cannula; and
a catheter connected to the spacing device and extendable through the cannula.

31. The system of claim 30 wherein each of the at least three inflatable chambers is adapted to receive a different flowable material.

32. The system of claim 30 wherein each of the at least three inflatable chambers is independently inflatable.

33. The system of claim 30 further comprising a pressure gauge for measuring the pressure in one of the at least three inflatable chambers.

34. A system for treating a nucleus pulposus of an intervertebral disc, the system comprising:
a cannula adapted to access an annulus fibrosus of the intervertebral disc;
a multi-chamber spacing device comprising two connected and inflatable chambers, wherein one of the inflatable chambers is expandable along the annulus fibrosus; and
a catheter connected to the spacing device and extendable through the cannula.

35. The system of claim 34 wherein the nucleus pulposus is unresected.

36. The system of claim 34 wherein the inflatable chamber expandable along the annulus fibrosus is adapted to contain a more rigid material than the other of the inflatable chambers.

37. The system of claim 34 wherein the inflatable chamber expandable along the annulus fibrosus is adapted to receive a resorbable material.

38. The system of claim 34 wherein the inflatable chamber expandable along the annulus fibrosus is adapted to occlude a defect in the annulus fibrosus.
